(19) United States
(12) Patent Application Publication
(10) Pub. No.: US 2026/0248742 A1
(43) Pub. Date: Aug. 27, 2026

(54) COMBINED PHARMACEUTICAL COMPOSITION OF SORBITOL AND PROGRAMMED CELL DEATH PROTEIN-1 INHIBITOR AND APPLICATION THEREOF

(71) Applicant: Huazhong University of Science and Technology Tongji Medical College Affiliated Union Hospital, Wuhan (CN)

(72) Inventors: Kaixiong TAO, Wuhan (CN); Yao LIN, Wuhan (CN); Yuping YIN, Wuhan (CN); Jianfeng SHEN, Wuhan (CN); Guopei ZHENG, Wuhan (CN)

(21) Appl. No.: 19/312,193

(22) Filed: Aug. 27, 2025

(30) Foreign Application Priority Data
Feb. 24, 2025 (CN) 202510199712.9

(51) Int. Cl. A61K 31/047; A61K 39/00; A61P 35/00; A61P 37/04; C07K 16/28

(52) U.S. Cl. CPC A61K 31/047; A61P 35/00; A61P 37/04; C07K 16/2818; A61K 2039/505; A61K 2039/545

(57) ABSTRACT

A combined pharmaceutical composition of sorbitol and a programmed cell death protein (PD)-1 inhibitor, as an anti-tumor combined pharmaceutical composition, includes sorbitol and a PD-1 inhibitor. The pharmaceutical composition is applied in the preparation of anti-tumor products. In the present disclosure, it is found for the first time that sorbitol has the indication of enhancing tumor immunotherapy, and sorbitol can be used in tumor immunotherapy to improve the survival and prognosis of tumor patients. Sorbitol combined with the PD-1 inhibitors can effectively kill tumor cells, which greatly improves the therapeutic effect of sorbitol compared with PD-1 inhibitors alone.

### CROSS REFERENCE TO RELATED APPLICATION

This patent application claims the benefit and priority of Chinese Patent Application No. 202510199712.9 filed with the China National Intellectual Property Administration on Feb. 24, 2025, the disclosure of which is incorporated by reference herein in its entirety as part of the present application.

### REFERENCE TO SEQUENCE LISTING

A computer readable XML file entitled “HKIP-US-1-1312-19-Sequence Listing”, that was created on Aug. 21, 2025, with a file size of about 21,343 bytes, contains the sequence listing for this application, has been filed with this application, and is hereby incorporated by reference in its entirety.

### TECHNICAL FIELD

The present disclosure belongs to the field of biomedical technology, and in particular relates to a combined pharmaceutical composition of sorbitol and a programmed cell death protein (PD)-1 inhibitor and an application thereof.

### BACKGROUND

Recombinant anti-PD-1 fully human monoclonal antibody blocks the binding of PD-1 and PD-L1 by high-affinity binding to PD-1 on the membrane of T lymphocytes, thereby enhancing and maintaining the activation state of the co-stimulatory signal of anti-tumor lymphocytes, promoting the activation of the anti-tumor effect of lymphocytes, activation and infiltration of tumor tissue, and ultimately killing tumor cells.

Currently, although immune checkpoint inhibitor therapy has made certain breakthroughs in multiple cancer types, the efficacy of single-agent immunization needs to be improved, especially in some solid tumors, including gastric cancer, intestinal cancer, and breast cancer. PD-1 inhibitors combined with chemotherapeutic drugs can improve the response rate and survival prognosis of tumor patients to a certain extent, but some patients still cannot benefit from it. The KEYNOTE-585 study is the first global phase III clinical study of immunotherapy combined with chemotherapy for gastric cancer to report survival data. In the main cohort, compared with the chemotherapy-alone group, the addition of pembrolizumab resulted in an improved pathologic complete response (pCR) rate in the short term (pCR rate: 12.9% vs. 2.0%), with an R0 resection rate reaching 80%. Although there is a trend toward benefit in long-term survival (Event-Free Survival (EFS) duration: 44.4 months vs. 25.3 months), it is not statistically significant, and longer follow-up is needed to confirm the survival outcomes.

The clinical indications of sorbitol include indigestion, abdominal distension, loss of appetite, constipation, intractable abdominal distension, and elderly habitual constipation. The sorbitol has minimal toxic and side effects and can be taken for long-term use. Meanwhile, sorbitol oral solution is inexpensive and commonly used clinically as an adjuvant medication to improve appetite.

There is an urgent need in this art to explore a more effective and safer combination drug regimen that synergizes with tumor immunotherapy.

### SUMMARY

In order to solve the above technical problems, the present disclosure, for the first time, reveals that sorbitol (in forms including powder, tablets, and injections) can enhance the immunotherapeutic effect of a PD-1 inhibitor, enhance anti-tumor immune function, and, based on this discovery, has developed a more effective combination drug regimen that synergizes with tumor immunotherapy to sensitize the efficacy of immunotherapy.

In order to achieve the above objective, the present disclosure adopts the following technical solutions.

The present disclosure provides an anti-tumor combined pharmaceutical composition, including sorbitol and a PD-1 inhibitor.

Further, a dosage form of the pharmaceutical composition is selected from injections, solutions, emulsions, suspensions, suppositories, ointments, creams, sprays, drops, powders, granules, electuary, capsules, pills, tablets, patches or sustained-release preparations.

Further, the sorbitol is administered once a week and the PD-1 inhibitor is administered once every three days.

Further, the PD-1 inhibitor is selected from nivolumab, toripalimab, pembrolizumab, sintilimab, camrelizumab or tislelizumab.

Further, the pharmaceutical composition further includes one or more pharmaceutically acceptable carriers or excipients.

In addition, the present disclosure also provides an application of the pharmaceutical composition in the preparation of anti-tumor products.

Further, the product is an anti-tumor drug.

Further, the anti-tumor drug further includes one or more pharmaceutically acceptable carriers or excipients.

Further, a dosage form of the anti-tumor drug is selected from injections, solutions, emulsions, suspensions, suppositories, ointments, creams, sprays, drops, powders, granules, electuary, capsules, pills, tablets, patches or sustained-release preparations.

Further, the tumor includes lung cancer, gastric cancer, colorectal cancer, endometrial cancer, liver cancer, breast cancer, melanoma, leukemia, glioma, cervical cancer, esophageal cancer or sarcoma.

Compared with the related art, the present disclosure has the following beneficial effects.

In the present disclosure, it is found for the first time that sorbitol has the indication of enhancing tumor immunotherapy, and sorbitol can be used in tumor immunotherapy to improve the survival and prognosis of tumor patients. Sorbitol combined with PD-1 inhibitors can effectively kill tumor cells, which greatly improves the therapeutic effect of sorbitol compared with PD-1 inhibitors alone.

In addition, in the present disclosure, it is further found for the first time that sorbitol is up-regulated in tumor patients sensitive to immunotherapy. The sorbitol can be used as a tumor biomarker for evaluating the efficacy of immunotherapy and provide a theoretical basis for clinical guidance of subsequent immunotherapy in tumor patients. At the same time, the application of sorbitol can further improve the situation where immunotherapy is not sensitive in patients with some tumor types.

### BRIEF DESCRIPTION OF THE DRAWINGS

FIG. 1 is a western blot (WB) image for detecting an expression of a key immunomodulatory factor cyclic GMP-AMP synthase (cGAS) in Example 1;

FIG. 2A is a quantitative polymerase chain reaction (qPCR) graph of immune factor transcription levels in MKN45 cell lines stimulated by sorbitol concentration;

FIG. 2B is a qPCR graph of immune factor transcription levels in CT26 cell lines stimulated by sorbitol concentration;

FIG. 3A is an in vitro protein sodium dodecyl sulfate-polyacrylamide gel electrophoresis (SDS-PAGE) image that after adding sorbitol, the transcriptional binding ability of a transcription factor SP1 of the key immunomodulatory factor cGAS is enhanced, verifying direct interaction;

FIG. 3B is a WB image that after adding sorbitol, the transcriptional binding ability of the transcription factor SP1 of the key immunomodulatory factor cGAS is enhanced, verifying direct interaction;

FIG. 4A is a schematic diagram of a binding between sorbitol and a metabolic enzyme Aldo-Keto Reductase Family 1 Member B1 (AKRIB1) thereof;

FIG. 4B is a schematic diagram of a binding between sorbitol and an AKRIB1-SP1 complex;

FIG. 5A is a titration graph of binding energy of a binding between AKRIB1 and transcription factors under sorbitol;

FIG. 5B is a titration curve of binding energy of a binding between AKRIB1 and the transcription factors under sorbitol;

FIG. 5C is a titration graph of binding energy of a binding between AKRIB1 and the transcription factors without sorbitol;

FIG. 5D is a titration curve of binding energy of a binding between AKRIB1 and the transcription factors without sorbitol; and combined with the above figures, sorbitol can enhance the ability of SP1 to regulate cGAS transcription;

FIG. 6A is a line graph of tumor growth volume in CT26 mice treated with sorbitol;

FIG. 6B is a scatter plot of sorbitol concentration in tumors of CT26 mice treated with sorbitol;

FIG. 7A is a schematic diagram of tumors of CT26 mice treated with aPD-1 immunotherapy;

FIG. 7B is a schematic diagram of tumors of CT26 mice treated with sorbitol combined with aPD-1 immunotherapy;

FIG. 7C is a scatter plot of tumor weight in CT26 mice treated with sorbitol;

FIG. 8A is a schematic diagram of tumors of 4T1 mice treated with sorbitol combined with aPD-1 immunotherapy;

FIG. 8B is a line graph of tumor growth volume in 4T1 mice treated with sorbitol;

FIG. 8C shows the concentration of sorbitol in tumors of 4T1 mice treated with sorbitol;

FIG. 9A is an illustration I of a patient tissue sample treated with sorbitol combined with aPD-1; and

FIG. 9B is an illustration II of the patient tissue sample treated with sorbitol combined with aPD-1.

### DETAILED DESCRIPTION

In order to better explain the present disclosure, specific examples are listed below. Obviously, the described examples are only a part of the present disclosure, rather than all of the examples. Based on the examples of the present disclosure, other examples obtained by those skilled in the art without creative efforts shall fall within the protection scope of the present disclosure.

The technical solutions of the present disclosure will be further described below through the accompanying drawings and examples.

Through extensive and in-depth research, it has been discovered for the first time that the combination of sorbitol and PD-1 inhibitors can significantly enhance the immunotherapeutic effects of PD-1 inhibitors, effectively treat tumors, and exhibit a synergistic effect. In addition, it can also effectively improve the clinical efficacy of existing immunotherapy regimens. On this basis, the present disclosure has been completed.

If specific techniques or conditions are not specified in the examples, the techniques or conditions described in documents in the art are used, or the product instructions are used. No reagents or instruments used are conventional products that can be purchased through regular channels if the surviving manufacturers are not specified.

The experimental methods in the following examples are all conventional methods unless otherwise specified. The test materials used in the following examples are commercially available products unless otherwise specified.

### Example 1: Effect of Adding Sorbitol on the Opening of Tumor Cell Immune Pathways

### 1.1 Cell Culture

Human gastric cancer cell MKN45 and mouse colorectal cancer cell CT26 were taken as examples.

Culture and passage of human gastric cancer cell MKN45 cell line and mouse colorectal cancer cell CT26 cell line: RPMI-1640 containing 10% fetal bovine serum (FBS) was taken as a culture medium and cultured in a cell culture incubator under conditions of saturated humidity, 37° C. and 5% CO2. The state of cells was observed every day. When the cells grew to 80-90%, the original culture solution was discarded, the cells were gently washed with phosphate-buffered saline (PBS), and were digested with 0.25% trypsin (containing ethylenediaminetetraacetic acid (EDTA)). Depending on the objective of the experiment, subculture was performed every day at a ratio of 1:2 or 1:3. After 3 passages, cells with good growth status and normal morphology and in a logarithmic growth phase were taken for experiments.

1.2 Cell plating: the cells in the logarithmic growth phase were taken, digested with 0.25% trypsin, blown evenly with RPMI-1640 cell culture solution containing 10% FBS, and dispersed into a single cell suspension. After counting, a concentration of the cell suspension was adjusted to 2-3×105/ml, 100 μL of the cell suspension was uniformly plated into each well of a 6-well plate, and incubated in an incubator with 37° C. and 5% CO2 with saturation humidity.

1.3 Dosing: after 24-48 h, until the cells grew to an appropriate density (80-90%), according to experimental needs, 3 duplicate wells were set up, sorbitol with concentrations of 0 mM, 1 mM, 2 mM, 4 mM, 8 mM, and 16 mM were added, and the cell plate after drug addition was continued to be incubated in an incubator with 37° C., 5% CO2 with saturated humidity for 72 h.

### 1.4 WB

Protein extraction, protein concentration determination (bicinchoninic acid (BCA) method) and Sodium Dodecyl Sulfate (SDS)-polyacrylamide gel electrophoresis (PAGE) were performed.

1.5 qPCR Experiment

Reagents and materials: template DNA or cDNA; forward and reverse primers; qPCR premixed solution (including DNA polymerase, deoxynucleoside triphosphates (dNTPs), buffer, etc.); SYBR Green or TaqMan probes (depending on experimental requirements); sterile water; qPCR reaction plates or tubes; and sealing film.

Instruments: qPCR instrument; centrifuge; pipette and sterile tips; and ice box.

Primer design: specific primers were designed using primer design software (e.g. Primer3 and NCBI Primer-BLAST). Primer length is typically 18-22 bp, GC content is 40-60%, and Tm value is 55-65° C. The finally obtained primer sequences are as shown in Table 1 below:

TABLE 1
Primer sequence list

| Target | Forward sequence | Reverse sequence |
|---|---|---|
| AKR1B1 | CAGCGCAACCAATCAGAAGG (SEQ ID | CTGCCCTGGAGGGGACTTTA |
| | NO. 1) | (SEQ ID NO. 2) |
| cGAS | ACATGGCGGCTATCCTTCTCT (SEQ ID | GGGTTCTGGGTACATACGTGAAA |
| | NO. 3) | (SEQ ID NO. 4) |
| CXCL8 | GTGCAGTTTTGCCAAGGAGT (SEQ ID | CTCTGCACCCAGTTTTCCTT |
| | NO. 5) | (SEQ ID NO. 6) |
| CXCL9 | TGATTGGAGTGCAAGGAACCC (SEQ ID | AATTTTCTCGCAGGAAGGGCT |
| | NO. 7) | (SEQ ID NO. 8) |
| IFNA1 | TCAAAGACTCTCACCCCTGC (SEQ ID | CAGTGTAAAGGTGCACATGACG |
| | NO. 9) | (SEQ ID NO. 10) |
| IFNA2 | CTTGTGCCTGGGAGGTTGTC (SEQ ID | AAAAGGTGAGCTGGCATACGA |
| | NO. 11) | (SEQ ID NO. 12) |
| IFNB | TCTCAAACTGAAGCTCGCACT (SEQ ID | TGGGGCATTGATTGCATCTGG |
| | NO. 13) | (SEQ ID NO. 14) |
| IFNG | GGGTTCTCTTGGCTGTTACTG (SEQ ID | TTTCTGTCACTCTCCTCTTTCC |
| | NO. 15) | (SEQ ID NO. 16) |
| β-ACTIN | CTCCATCCTGGCCTCGCTGT (SEQ ID | GCTGTCACCTTCACCGTTCC |
| | NO. 17) | (SEQ ID NO. 18) |

Reaction system configuration: reaction system (20 μL as an example), qPCR premixed solution 10 μL, forward primer (10 μM) 0.8 μL, reverse primer (10 μM) 0.8 μL, DNA/cDNA template 2 μL, and sterile water 6.4 μL.

Reaction program setting: pre-denaturation: 95° C., 5 min.

Amplification cycles (40 cycles): denaturation: 95° C., 15 s, annealing: 55-60° C., 30 s (adjusted according to primer Tm value), and extension: 72° C., 30 s.

Melting curve analysis (SYBR Green experiment): 95° C., 15 s, 60° C., 1 min, 95° C., 15 s.

### 1.6 Experimental Results

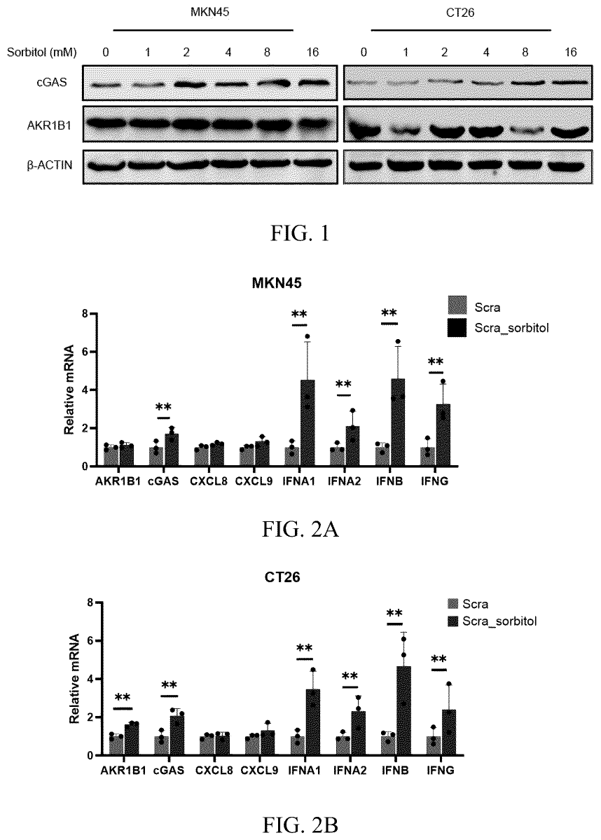

As shown in FIG. 1, adding sorbitol with different concentrations can significantly improve the protein of cGAS, a key immunoregulatory factor in human gastric cancer cell MKN45 cell line and mouse colorectal cancer cell CT26 cell line. At the same time, as shown in FIG. 2A and FIG. 2B, qPCR experiments suggest that sorbitol can significantly increase the transcription levels of downstream immune factors CXCL8, CXCL9, IFNA1, IFNA2 and IFNG in tumor cells, and open up immune pathways.

This experiment successfully demonstrated that sorbitol can open the immune pathway in tumor cells through cGAS at the cellular level. Sorbitol can improve the immunosuppression level of tumor cells.

### Example 2 Exploration of the Mechanism of Sorbitol Regulating Immune Pathways In Vivo in Tumor Cells

### 2.1 Experimental Materials

Drugs: sorbitol; cell line: human gastric carcinoma cell MKN45 cell line.

### 2.2 Experimental Methods

### 2.2.1 Protein-Protein Interaction Experiment (Co-Immunoprecipitation (Co-IP))

(1) Sample preparation: firstly, samples of cells or tissues were collected, washed with precooled PBS buffer for three times, and immunoprecipitation (IP) lysis buffer was added for lysis. Protease inhibitors were added during lysis to prevent protein degradation. After lysis, the samples were centrifuged at 4° C. and the supernatant was collected for later use.
(2) Binding of antibody and protein: the processed magnetic beads were mixed with antibody, and incubated on a tumbling mixer. After incubation, the magnetic beads were collected by magnetic separation and washed several times with binding/washing buffer to remove non-specifically binding proteins. Binding of antigen and antibody: the target protein was mixed with the magnetic beads, and after further incubation, the magnetic beads were magnetically separated and washed to remove unbinding protein.
(3) Protein isolation and detection: a mixture of magnetic beads and protein sample was separated by SDS-PAGE electrophoresis, and the presence of the target protein was detected by WB. If bait protein and target protein can be detected in the precipitate, it is indicated that there is an interaction between the two proteins.
(4) Experimental principle: the Co-IP experiment is based on the specific interaction between antibody and antigen, and is a classic method used to study protein interactions. The protein binding to a specific target protein is indirectly captured by using a target protein-specific antibody to form an antibody-target protein complex, the complex is immobilized and precipitated using a bead-like support capable of binding to the antibody, and finally analyzed by SDS-PAGE and WB.

### 2.2.2 Glutathione S-Transferase (GST)-Pulldown Experiment

### (1) Protein Extraction

In step 1), washing: the sample (approximately 2×107 cells) was washed twice with 1 ml of precooled PBS, and the PBS was dried as much as possible for the last time.

In step 2), lysis: according to the amount of cells, 990 mL Lysis buffer, 10 μL Protease Inhibitor (100×) were added, fully performed lyse on ice for 20-30 min, and mixed upside down every 5 min.

In step 3), ultrasound: an ultrasonic cell disruptor is used for 5 min, with a power of 20%, ultrasonic for 3 s, intermittent for 3 s, and ultrasonic in ice bath.

In step 4), centrifugation: 4° C., 12000 rpm, 10 min, and supernatant was collected.

### (2) Magnetic Bead Preparation

In step 1), the Glutathione MagBeads were taken out from a refrigerator at 4° C., and inverted for several times to mix the magnetic beads and the solution evenly; and 30 μL of the Glutathione MagBeads were taken into 2 clean 1.5 mL EP tubes, and recorded as a control group and an experimental group.

In step 2), 0.5 mL of precooled Washing buffer was added to resuspend the magnetic beads, stood on a magnetic rack for 1 min, the magnetic beads and the solution were separated, and supernatant was carefully sucked and discarded with a pipette.

In step 3), step 2) was repeated for 2 times for a total of 3 washes.

### (3) Magnetic Beads Binding GST-X Protein

In step 1), 200 ug of GST-X recombinant protein was added to the experimental group, no protein was added to the control tube or 200 ug of GST recombinant protein was added, volumes were supplemented to 1 mL with Incubation buffer, and mixtures were incubated for 2 h at room temperature.

In step 2), 2 tubes were placed on the magnetic rack and stand for 1 min, the magnetic beads and the solution were separated, and supernatant was carefully sucked and discarded with the pipette.

In step 3), 0.5 mL of precooled Washing buffer was added, and stood on the magnetic stand for 1 min, the magnetic beads and the solution were separated, and supernatant was carefully sucked and discarded with the pipette.

In step 4), step 3) was repeated for 2 times for a total of 3 washes.

### (4) Bait Protein Binding Interaction Protein Y (Other Recombinant Proteins or Total Proteins Except GST Tag)

In step 1), 200 ug Y recombinant protein or 500-1000 ug total protein were added to 2 tubes, volumes were supplemented to 1 mL with Incubation buffer, and mixtures were incubated overnight (about 16 h) at 4° C. with silent mixing.

In step 2), 2 tubes were placed on the magnetic rack and stood for 1 min, the magnetic beads and the solution were separated, and supernatant was carefully sucked and discarded with the pipette.

In step 3), 0.5 mL precooled Washing buffer was added, stood on the magnetic stand for 1 min, the magnetic beads and the solution were separated, and supernatant was carefully sucked and discarded with the pipette.

In step 4), step 3) was repeated for 2 times for a total of 3 washes.

### (5) Elution

In step 1), 100 uL of eluent was added to both 2 tubes, mixtures were boiled for 10 min and centrifuged at 12000 rpm for 5 min, supernatant was taken, the mixtures were added with 20 uL 6× Loading buffer and boiled for 8-10 min, which were recorded as a control group and an experimental group.

In step 2), another 2 clean 1.5 ml EP tubes were taken, and recorded as an Input control group and an Input experimental group; and 50 ug of GST recombinant protein and Y recombinant protein were added into the Input control group, 50 ug of GST-X protein and Y recombinant protein were added into the Input experimental group (for total protein, 100 uL was added), ⅕ volume of 6× Loading buffer was add, and boiling water bath is carried out for 8-10 min.

In step 3), the control group, experimental group, Input control and Input experiment were stored at −20° C. for later use.

(6) WB: each 30 μL mixture of the control group, experimental group, Input control group and Input experimental group was taken for SDS-PAGE and WB detection.

### 2.3 Experimental Results

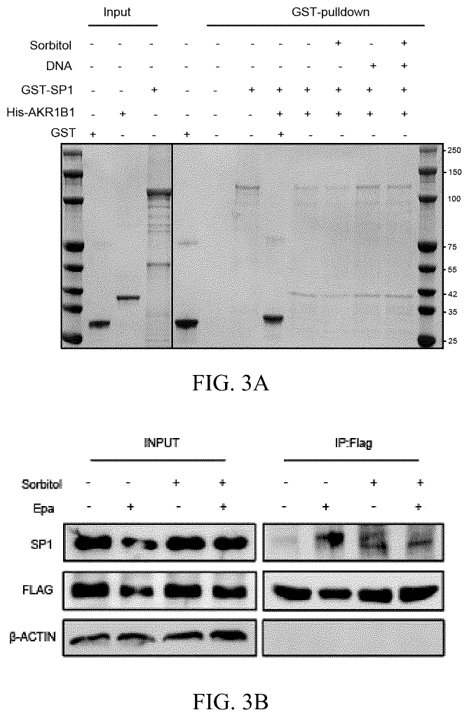

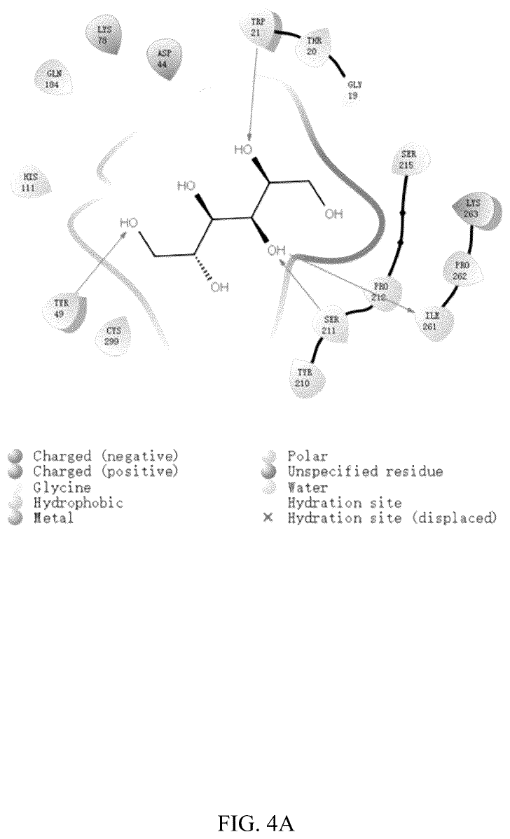

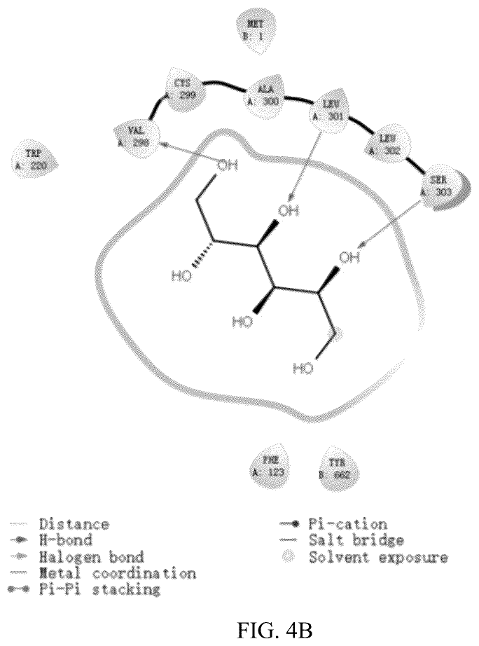

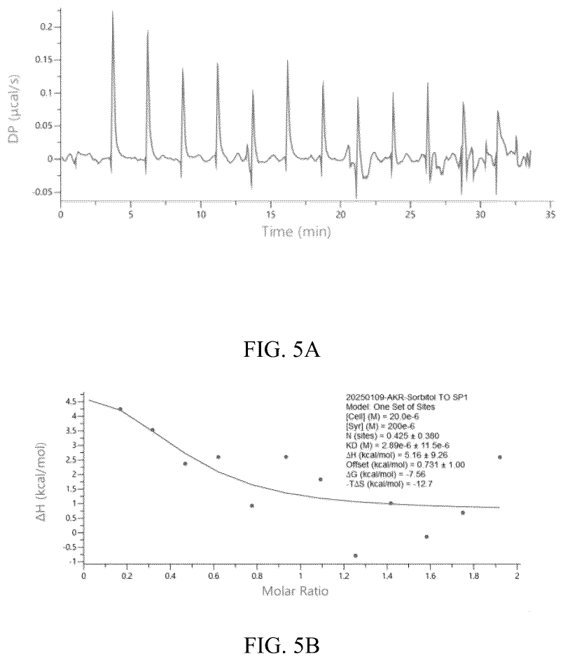

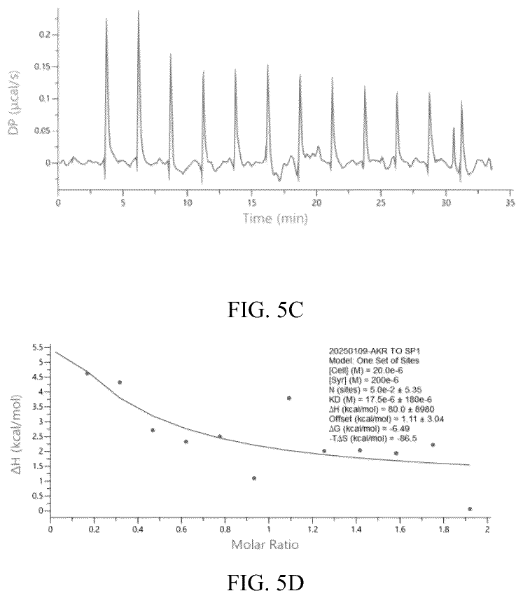

As shown in FIG. 3A and FIG. 3B, SP1 protein is known to be a transcription factor of cGAS. Through Co-IP experiment and GST-Pulldown experiment, it was found that adding sorbitol could enhance the binding of metabolic enzyme AKRIB1 with SP1 and improve the transcription level of cGAS. At the same time, as shown in FIG. 4A and FIG. 4B, sorbitol can bind to the AKRIB1-SP1 complex and form stable hydrogen bonds, enhancing its stability. In addition, as shown in FIGS. 5A-5D, it was further found that sorbitol could reduce the binding energy of AKRIB1 and SP1, making it easier to bind.

It can be seen from this that the addition of sorbitol can enhance the binding ability of AKRIB1 to transcription factor SP1, and further enhance the transcription level of cGAS, thereby opening the immune pathway.

### Example 3 In Vivo Pharmacodynamic Study of Combination Therapy on Solid Cancers in Mice

### 3.1 Experimental Materials

Drugs: anti-PD-1 antibody, product information: Anti-Mouse CD279 (PD-1) (Clone RMP1-14)-Purified in vivo GOLD Functiona 1 Grade (USA); and brand: Leinco; and
sorbitol; cell lines: mouse breast cancer cells 4T1, mouse colorectal cancer cells CT26; and animals: Bal/B mice.

### 3.2 Experimental Methods

### 3.2.1 Dosing Preparation Configuration

Sorbitol (5% kg/L) was added to the daily diet of mice.

### 3.2.2 Mouse Breast Cancer Cell 4T1 Culture:

Mouse breast cancer cells 4T1 were cultured in a 1640 medium containing 10% FBS or DMEM/F12 medium containing 10% FBS, and passaged twice a week. On the day of inoculation of mice, the cells were treated with trypsin, collected in a 50 ml centrifuge tube, centrifuged to precipitate, supernatant was discarded, the cells were washed twice with PBS, and finally suspended in PBS at a cell density of 5×106 cells/ml.

### 3.2.3 Mouse Colorectal Cancer Cell CT26 Culture:

Mouse colon cancer cells CT26 were cultured in a 1640 medium containing 10% FBS or DMEM/F12 medium containing 10% FBS, and passaged twice a week. On the day of inoculation of mice, the cells were treated with trypsin, collected in a 50 ml centrifuge tube, centrifuged to precipitate, supernatant was discarded, the cells were washed twice with PBS, and finally suspended in PBS at a cell density of 5×106 cells/ml.

### 3.2.4 Subcutaneous Transplantation of Murine Breast Cancer Cells 4T1 in Mice

The collected cells were placed on ice and brought to a mouse breeding facility. Each mouse is injected subcutaneously with 0.1 ml of cell suspension containing 5×105 cells on the back.

3.2.5 Subcutaneous transplantation of murine colorectal cancer cells CT26 in mice

The collected cells were placed on ice and brought to a mouse breeding facility. Each mouse is injected subcutaneously with 0.1 ml of cell suspension containing 5×105 cells on the back.

### 3.2.6 Mouse Administration

The administration method was intraperitoneal injection, and the administration volume was 0.2 ml per mouse. At Day8, Day12, Day15 and Day19, antiPD-1 was administered four times a day. The mouse body weight and tumor volume were measured once every 2-3 days starting from D5 (the body weight and tumor volume were measured before administration during the administration), (tumor volume V (mm3)=½×tumor long diameter a (mm)×tumor short diameter b2 (mm)). Specific experimental groupings are shown in Table 2.

TABLE 2
Dose grouping of each group

| | | Types of admin- | | | | |
|---|---|---|---|---|---|---|
| | | istration and dosage | | | | |
| Group | | | | PBS | Number | Dosing |
| Tumor type | Category | antiPD-1 | Sorbitol | (μl/dose) | of mice | time points |
| Colorectal | Negative | | | 200 | The control | AntiPD-1 was |
| cancer | control group | | | | group and the | administered |
| | Experimental | 200 μg | | | experimental | every three days |
| | group 1 | | | | group were 7 | for a total of 5 |
| | Experimental | | Drinking | | female mice | doses. After |
| | group 2 | | water | | per group. | inoculation, the |
| | | | added 5% | | | tumor was given |
| | | | (kg/L) | | | 100-200 mm³ for |
| | Experimental | 200 μg | Drinking | | | the first time and |
| | group 3 | | water | | | injected |
| | | | added 5% | | | intraperitoneally. |
| | | | (kg/L) | | | Sorbitol was |
| Breast | Control group | 200 μg | | | | added to drinking |
| cancer | Experimental | 200 μg | Drinking | | | water, and 200 mL |
| | group 1 | | water | | | of water was |
| | | | added 5% | | | changed once a |
| | | | (kg/L) | | | week. |

### 3.2.7 Statistics and Analysis

The mouse body weight data, tumor volume, and tumor weight data of the experimental group and the control group were expressed in a form of (arithmetic mean±standard error). T-test analysis is carried out on the data between groups to determine whether the data between groups are significantly different.

### 3.2.8 Mouse Tumor Volume Monitoring Results

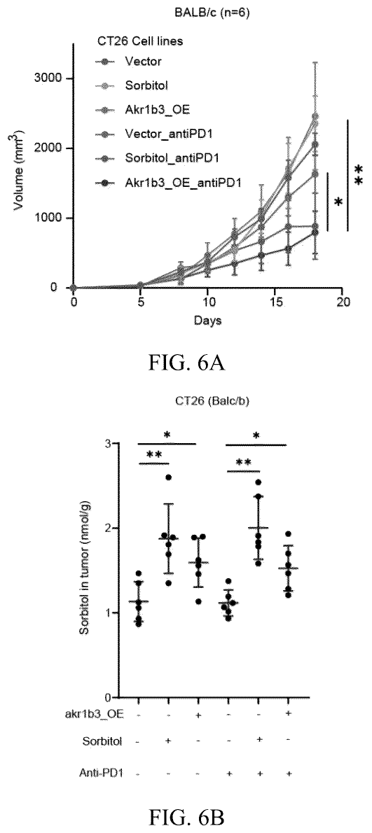

FIG. 6A and FIG. 6B show changes in the average volume of colorectal cancer tumors of mice in each group during the experiment. It can be clearly seen that after Day 12, the tumors of the control group, the sorbitol only added group and the mice treated only with antiPD-1 increased significantly, and the tumors of the mice treated with sorbitol and antiPD-1 were significantly inhibited in growth, with statistically significant differences (P<0.05). T-test analysis was carried out on the individual tumor volume data of mice in the combination administration group and the antiPD-1 monotherapy group. The results showed that there was also a statistically significant difference between the combination administration group and the single-drug treatment group (P<0.05). Sorbitol was also detected in dissected tumor tissues. The results showed that the concentration of sorbitol in the tumor tissues of the mouse group fed sorbitol increased significantly, which was statistically significant (P<0.05).

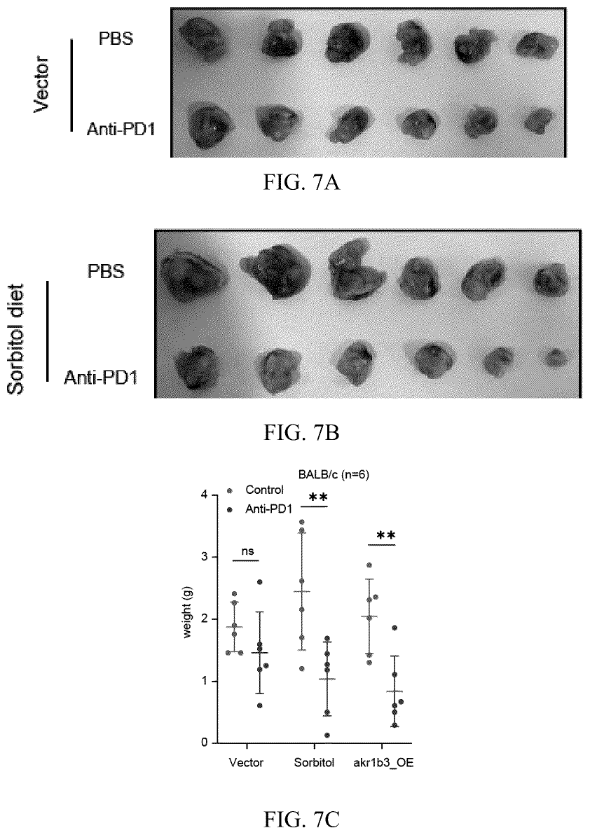

FIGS. 7A to 7C show photographs of colorectal cancer tumors stripped from each group of mice and comparison of body weight. Compared with the PBS group, the tumor volume of mice in the combination drug group showed a statistically significant difference (P<0.05). At the end of the experiment on Day20, the average tumor volume in each group of mice is shown in Table 3 below:

TABLE 3
Average volume of colorectal cancer tumors in each group of mice

| Group name | Average tumor volume in mice (mm³) |
|---|---|
| PBS group | 1987.34 |
| antiPD-1 single drug group | 1354.98 |
| Sorbitol group | 1998.98 |
| Sorbitol + antiPD-1 group | 897.12 |
| AKR1B1 overexpression group | 1992.31 |
| AKR1B1 overexpression + | 734.19 |
| antiPD-1 group | |

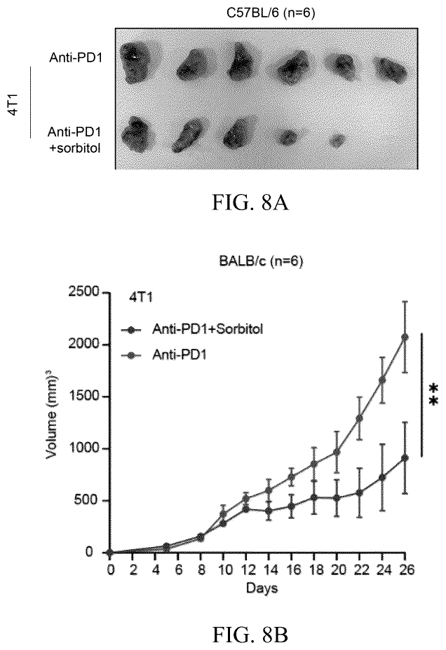

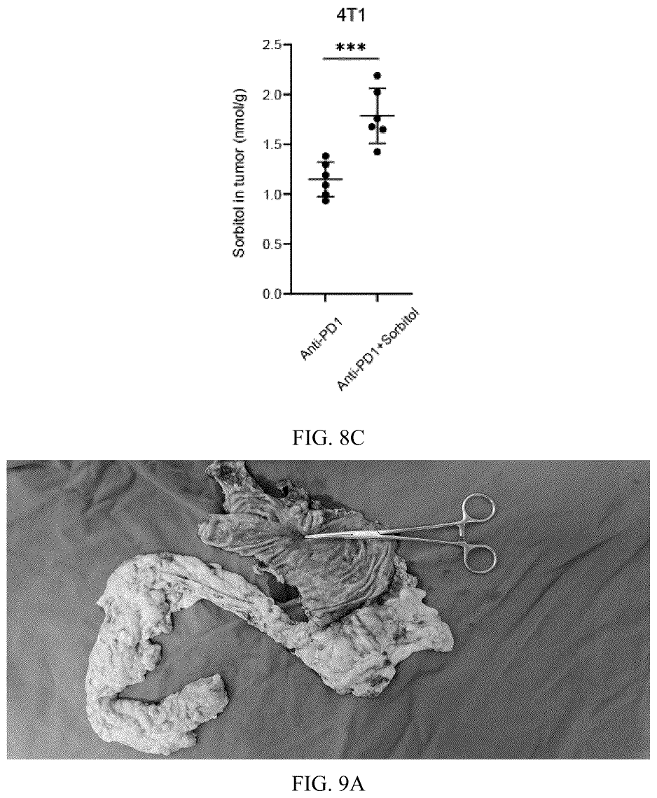

In order to further explore the role of sorbitol in other tumors, it was verified in a mouse breast cancer tumor model, as shown in FIGS. 8A-8C. Compared with the anti-PD-1 single drug group, the tumor size of mice in the combination treatment group was significantly inhibited, and even the tumor of one mouse disappeared.

In this experiment, a combination of feeding 5% sorbitol in drinking water and intraperitoneal injection of antiPD-1 was administered to colorectal cancer CT26 and breast cancer 4T1 once every three days for a total of 5 times. At the same time, a single administration group of antiPD-1 and 5% sorbitol and a PBS negative control group were set up to examine whether the anti-tumor effect of antiPD-1 and sorbitol in mice colorectal cancer CT26 and breast cancer 4T1 in vivo has a synergistic effect. Monotherapy of antiPD-1 (intraperitoneal injection) had a certain effect on tumor growth, but there was no significant statistical difference. Sorbitol alone had no significant effect on tumor growth. After the combined application of sorbitol and antiPD-1, mouse tumors showed a synergistic effect of sorbitol and antiPD-1 in inhibiting tumor growth.

### Example 4 Preliminary Application of a Clinical Treatment Regimen in which Sorbitol Sensitizes the Efficacy of PD-1 Inhibitors (this Study has Passed the Ethics Review of this Unit)

Overall design: this study is an exploratory investigation based on the concepts of tumor metabolism and immunity on the efficacy of oral sorbitol-enhanced SOX chemotherapy regimens combined with tislelizumab (PD-1 inhibitor) for neoadjuvant treatment of advanced gastric/gastroesophageal junction adenocarcinoma. The study was divided into 3 stages: a screening period, a treatment period (neoadjuvant therapy, surgery and adjuvant therapy periods), and a follow-up period. Forty subjects were enrolled in this study. The enrolled subjects were randomly divided into two groups. Group A: oral administration of sorbitol+PD1 inhibitor+SOX for 3 cycles (1 cycle=every 3 weeks [Q3W]); and Group B: placebo+PD1 inhibitor+SOX for 3 cycles (1 cycle=every 3 weeks [Q3W]).

In the above clinical protocol, all patients signed informed consent and passed unit ethics.

Nivolumab, toripalimab, pembrolizumab, sintilimab, camrelizumab or tislelizumab may be selected as the PD-1 inhibitor during this experiment.

Study objectives: the safety and efficacy (major pathologic response rate, MPR) of neoadjuvant chemotherapy combined with tislelizumab (PD-1 inhibitor) in locally advanced gastric cancer patients with oral sorbitol.

Study subjects: forty patients with advanced gastric/gastroesophageal junction adenocarcinoma were enrolled in this study.

Specific research methods:
(1) Screening period: the subjects are screened and evaluated within 14 days before the first dosing to determine whether the subjects meet the research conditions. Specific conditions: after histological confirmation, the patients have untreated HER2-negative gastric cancer or gastroesophageal junction (GEJ) cancer, with a clinical stage of cT3-4N+M0, and histological examination has confirmed that the tumors are predominantly adenocarcinomas. Only subjects with Siewert type III GEJ cancer and those with Siewert type II GEJ cancer not requiring combined thoracotomy are eligible for enrollment. For patients who meet the inclusion and exclusion criteria, routine blood tests, imaging examinations, and Helicobacter pylori tests are to be completed, followed by further molecular-level testing. This includes assessing the microsatellite instability (MSI) status, PD-L1 expression combined positive score (CPS), and Epstein-Barr virus-encoded RNA (EBER) detection in pre-treatment biopsy pathological tissues.
(2) Neoadjuvant (pre-operative) period: 3 cycles

The enrolled subjects were randomly divided into two groups.
Group A (sorbitol complementary diet group): PD-1 inhibitor+SOX for 3 cycles (1 cycle=every 3 weeks [Q3W]); and during the neoadjuvant therapy cycles, sorbitol was administered orally 2-4 g/time, three times daily (with meals).
Group B (placebo complementary diet group): PD-1 inhibitor+SOX for 3 cycles (1 cycle=every 3 weeks [Q3W]); and during the neoadjuvant therapy cycles, placebo was administered orally 2-4 g/time, three times daily (with meals). The placebo is glucose solution.

### (3) Surgery: After Evaluation of Neoadjuvant Therapy

After neoadjuvant therapy, for subjects deemed eligible for surgery based on the study's assessment, surgery is performed within 2-6 weeks after the last dose of neoadjuvant therapy (including oral medications). For subjects who are not candidates for surgery, the investigator will make a comprehensive determination based on clinical circumstances to determine the subsequent treatment plan. A small amount of pathological tissue is collected during surgery, and testing for AKRIB1/cGAS targets and sorbitol content is conducted.

### (4) Adjuvant Therapy (Postoperative) Period: 5 Cycles

Adjuvant therapy phase: after postoperative unblinding, adjuvant therapy can be initiated 3 to 12 weeks after operation (beyond 12 weeks, the investigator may decide whether to continue using the investigational drug). Subjects in Groups A and B were grouped according to the tumor regression grade (TRG) classification. Those with a TRG score of 0-1 continued with the original treatment regimen, while those with a TRG score greater than 2 received SOX chemotherapy alone for a maximum of 5 cycles, or until the investigator determined that the subject had lost clinical benefit, died, experienced intolerable toxicity, withdrew informed consent, or for other reasons specified in the protocol (whichever occurred first).

### (5) Follow-Up Period:

Safety follow-up and survival follow-up are included. After the completion of treatment, all subjects will be followed up. For subjects who discontinue treatment for reasons other than progressive disease (PD), follow-up will be conducted at the originally planned frequency until the occurrence of PD, withdrawal of informed consent, or loss to follow-up (whichever occurs first). After the completion of treatment and safety follow-up, survival follow-up will be conducted for all subjects (OS data collected every 3 months±14 days) until death, withdrawal of informed consent, loss to follow-up, or study termination (whichever occurs first).

The preliminary results of the study are shown in Table 4 below:

TABLE 4
Clinical treatment solution preliminary study results

| | Tumor regression grade (TRG) | | |
|---|---|---|---|
| Group | TRG = 0 | TRG = 1 | TRG = 2&3 |
| Sorbitol complementary | 6 (30%) | 11 (55%) | 3 (15%) |
| diet group | | | |
| (n = 20) | | | |
| Placebo complementary | 3 (15%) | 9 (45%) | 8 (40%) |
| diet group | | | |
| (n = 20) | | | |

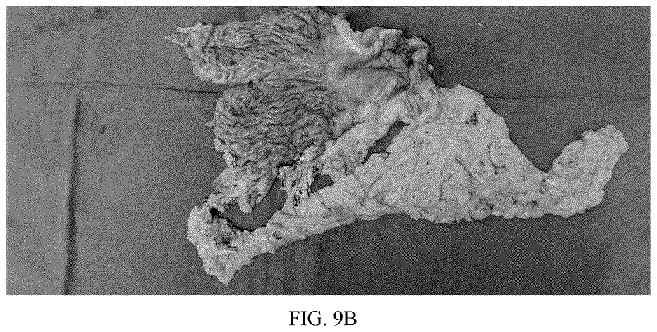

FIGS. 9A and 9B present the treatment results of two gastric cancer patients: both patients exhibited no significant adverse reactions during treatment and achieved major pathological response (MPR) (patient 1 with TRG=1; and patient 2 with TRG=0).

The final pathological report of patient 1 indicates the following: 1. (Post-neoadjuvant therapy for gastric malignancy initially biopsied at an external hospital) (Gastroesophageal junction) Well-differentiated adenocarcinoma—Tumor invasion into the muscularis mucosae, with mucus visible in the submucosa and no atypical epithelial cells observed-Pathological staging: ypT1a—Lymphovascular invasion (−)—Perineural invasion (−)—No cancer metastasis detected in any of the lymph nodes (0/29). Grouping description: No cancer metastasis was observed on the histological sections of the pericardiac lymph node (1 node), (Groups 1 and 3) lymph nodes (8 nodes), (Group 2) lymph node (1 node), (Group 4) lymph nodes (3 nodes), (Groups 7, 8, and 9) lymph nodes (12 nodes), (Group 10) lymph node (1 node), (Groups 8 and 11) lymph nodes (2 nodes), and (Groups 9 and 12) lymph node (1 node). —Margins: esophageal margins (−) gastric corpus margins (−) esophageal (proximal) margins (−) omentum (−) immunohistochemical staining showed tumor cells: P53 (partial+, two wax blocks were detected). Tumor TRG grading after neoadjuvant therapy: grade 1 (moderate reaction, graded 0-3) 2. Chronic cholecystitis with gallstones. (Other consultation specimen pathology No.: CI24-09390)

Patient 2 was clinically diagnosed with leather stomach, a type of gastric cancer that is extremely insensitive to immunotherapy, and achieved complete remission after the application of a new regimen of sorbitol combined with immunotherapy, and the final pathological results showed that after neoadjuvant chemotherapy for gastric cancer (antral and gastric body), there was no definite cancer residue in the original tumor bed under the microscope, and multiple mucus lakes were formed, the deepest part was located in the subserosal layer, and floating chronic inflammatory cells could be seen in the mucus lake. —No intravascular thrombosis and nerve invasion: —No cancer involvement was found in the portal margin, pyloric margin, esophageal margin and omentum: —(3 groups) of the bat node (⅕ of the large and (4 groups) lymph node ( 4/14) of the lymph node ( 4/14) showed mucus lake formation, and no viable tumor cells. No metastasis was found in (Groups 1) lymph nodes (5 nodes), (Groups 5 and 6) lymph node (3 nodes), (Group 2) lymph nodes (5 nodes), (Groups 7, 8, and 9) lymph nodes (2 nodes), (Group 11) lymph node (3 nodes), and (Groups 12) lymph nodes (4 nodes). The tumor TRG grade is 0 (no cancer cells remaining, complete response, and consultation specimen pathology No.: CI24-09201).

This study demonstrates that sorbitol combined immunotherapy has excellent safety and efficacy in humans.

In summary, it is the first time to find that sorbitol and antiPD-1 immune preparation have synergistic anti-tumor effects. Sorbitol can be used to treat cancer patients, combined with immunotherapy has promising therapeutic prospects.

The above-mentioned examples merely describe the preferred ways of the present disclosure and do not limit the scope of the present disclosure. Various modifications and improvements made to the technical solutions of the present disclosure by those skilled in the art without departing from the spirit of the present disclosure shall fall within the scope of protection determined by the claims of the present disclosure.

1. An anti-tumor combined pharmaceutical composition, comprising sorbitol and a programmed cell death protein (PD)-1 inhibitor.

2. The pharmaceutical composition according to claim 1, wherein a dosage form of the pharmaceutical composition is selected from injections, solutions, emulsions, suspensions, suppositories, ointments, creams, sprays, drops, powders, granules, electuary, capsules, pills, tablets, patches or sustained-release preparations.

3. The pharmaceutical composition according to claim 1, wherein the sorbitol is administered once a week and the PD-1 inhibitor is administered once every three days.

4. The pharmaceutical composition according to claim 1, wherein the PD-1 inhibitor is selected from nivolumab, toripalimab, pembrolizumab, sintilimab, camrelizumab or tislelizumab.

5. The pharmaceutical composition according to claim 1, further comprising one or more pharmaceutically acceptable carriers or excipients.


SEQUENCE LISTING (18 sequences)

SEQ ID NO: 1 - DNA, 20 bp, synthetic construct
  source 1..20: mol_type = other DNA; note = Primer sequence for amplification of the target AKR1B1, forward sequence
cagcgcaaccaatcagaagg

SEQ ID NO: 2 - DNA, 20 bp, synthetic construct
  source 1..20: mol_type = other DNA; note = Primer sequence for amplification of AKR1B1, reverse sequence
ctgccctggaggggacttta

SEQ ID NO: 3 - DNA, 21 bp, synthetic construct
  source 1..21: mol_type = other DNA; note = Primer sequence for amplification of the target cGAS, forward sequence
acatggcggctatccttctct

SEQ ID NO: 4 - DNA, 23 bp, synthetic construct
  source 1..23: mol_type = other DNA; note = Primer sequence for amplification of the target cGAS, reverse sequence
gggttctgggtacatacgtgaaa

SEQ ID NO: 5 - DNA, 20 bp, synthetic construct
  source 1..20: mol_type = other DNA; note = Primer sequence for amplification of the target CXCL8, forward sequence
gtgcagttttgccaaggagt

SEQ ID NO: 6 - DNA, 20 bp, synthetic construct
  source 1..20: mol_type = other DNA; note = Primer sequence for amplification of the target CXCL8, reverse sequence
ctctgcacccagttttcctt

SEQ ID NO: 7 - DNA, 21 bp, synthetic construct
  source 1..21: mol_type = other DNA; note = Primer sequence for amplification of the target CXCL9, forward sequence
tgattggagtgcaaggaaccc

SEQ ID NO: 8 - DNA, 21 bp, synthetic construct
  source 1..21: mol_type = other DNA; note = Primer sequence for amplification of the target CXCL9, reverse sequence
aattttctcgcaggaagggct

SEQ ID NO: 9 - DNA, 20 bp, synthetic construct
  source 1..20: mol_type = other DNA; note = Primer sequence for amplification of the target IFNA1, forward sequence
tcaaagactctcacccctgc

SEQ ID NO: 10 - DNA, 22 bp, synthetic construct
  source 1..22: mol_type = other DNA; note = Primer sequence for amplification of the target IFNA1, reverse sequence
cagtgtaaaggtgcacatgacg

SEQ ID NO: 11 - DNA, 20 bp, synthetic construct
  source 1..20: mol_type = other DNA; note = Primer sequence for amplification of the target IFNA2, forward sequence
cttgtgcctgggaggttgtc

SEQ ID NO: 12 - DNA, 21 bp, synthetic construct
  source 1..21: mol_type = other DNA; note = Primer sequence for amplification of the target IFNA2, reverse sequence
aaaaggtgagctggcatacga

SEQ ID NO: 13 - DNA, 21 bp, synthetic construct
  source 1..21: mol_type = other DNA; note = Primer sequence for amplification of the target IFNB, forward sequence
tctcaaactgaagctcgcact

SEQ ID NO: 14 - DNA, 21 bp, synthetic construct
  source 1..21: mol_type = other DNA; note = Primer sequence for amplification of the target IFNB, reverse sequence
tggggcattgattgcatctgg

SEQ ID NO: 15 - DNA, 21 bp, synthetic construct
  source 1..21: mol_type = other DNA; note = Primer sequence for amplification of the target IFNG, forward sequence
gggttctcttggctgttactg

SEQ ID NO: 16 - DNA, 22 bp, synthetic construct
  source 1..22: mol_type = other DNA; note = Primer sequence for amplification of the target IFNG, reverse sequence
tttctgtcactctcctctttcc

SEQ ID NO: 17 - DNA, 20 bp, synthetic construct
  source 1..20: mol_type = other DNA; note = Primer sequence for amplification of the target beta-ACTIN, forward sequence
ctccatcctggcctcgctgt

SEQ ID NO: 18 - DNA, 20 bp, synthetic construct
  source 1..20: mol_type = other DNA; note = Primer sequence for amplification of the target beta-ACTIN, reverse sequence
gctgtcaccttcaccgttcc